(19) United States
(12) Patent Application Publication
(10) Pub. No.: US 2007/0059297 A1
(43) Pub. Date: Mar. 15, 2007

(54) Methods and compositions for improving visual acuity

(75) Inventors: Mark K. Waldron, Savigny (CH); John E. Bauer, College Station, TX (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Nestec, S.A.

(21) Appl. No.: 11/499,044

(22) Filed: Aug. 3, 2006

Related U.S. Application Data
(60) Provisional application No. 60/705,510, filed on Aug. 4, 2005.

(51) Int. Cl. A61K 35/74; A61K 31/202; A61K 31/015

(52) U.S. Cl. 424/93.45; 514/560; 424/442; 514/763; 514/456

(57) ABSTRACT

Compositions and methods for enhancing visual acuity in animals are disclosed. The compositions and methods utilize long chain polyunsaturated fatty acids.

This claims benefit of U.S. Provisional Application No. 60/705,510, filed Aug. 4, 2005, the entirety of which is incorporated by reference herein.

### FIELD OF THE INVENTION

The present invention is related to mammalian nutrition and effects thereof on vision. In particular, the present invention utilizes long chain polyunsaturated fatty acids, administered during gestation through the maternal diet, or post-parturition from maternal milk or directly through diet as the animal matures, to improve visual acuity.

### BACKGROUND OF THE INVENTION

Various publications, including patents, published applications, technical articles and scholarly articles are cited throughout the specification. Each of these cited publications is incorporated by reference herein, in its entirety. Full citations for publications not cited fully within the specification are set forth at the end of the specification.

Both (n-3) and (n-6) classes of long-chain polyunsaturated fatty acids (LCPUFA) are important in perinatal development. Increasing evidence indicates that the (n-3) fatty acids are of particular importance in neural and retinal development. In primates, neural development begins in the third trimester of gestation, peaks about the time of birth, and continues for about 18-24 months after parturition (Menard, C R et al. 1998, Martinez, M 1992). Although differences are likely, it is believed that this pattern of development holds true among most mammalian species. (Bauer J E et al. 2004).

During this developmental period, fatty acids such as arachidonic acid (AA) and docosahexaenoic acid (DHA) are rapidly incorporated into the neural tissues (Sinclair, A J 1975, Greiner, R C et al. 1997). Accumulation of DHA occurs primarily during late gestation and in the postnatal period of development, although enrichment of DHA into neurological tissues continues post parturition (Carnielli, V P et al. 1998). DHA is primarily found in the serine and ethanolamine phospholipids in retinal and neurological tissue. In the retina, DHA is highly conserved, especially during periods of n-3 fatty acid deficiency.

The incorporation of supplemental DHA into neurological and retinal tissue has been investigated. In vitro studies showed that rat retina neuronal cells incubated with DHA had four- to six-fold more DHA than cells incubated with other fatty acids (Rotstein, N P et al. 1999). The addition of other fatty acids in that study had no effect on altering cell membrane fatty acid compositions. The report suggested that retinal neurons have specific mechanisms for handling fatty acids of different length and desaturation and the selective uptake DHA. Indeed, there appears to be at least one mechanism by which DHA is selectively taken up by neural and retinal tissues. Studies in pigs showed that diets supplemented with DHA increased brain accumulation of DHA during the postnatal growth period (Morris S A et al. 1999). In addition, in vivo studies have shown that supplemental DHA is accumulated into neurological and retinal tissues in piglets, kittens, and non-human primates (Pawlosky, R J et al. 1997, Green, P et al. 1996). Conversely, a deficiency of DHA has been shown to be deleterious in laboratory species. Rats and Guinea pigs fed diets deficient in n-3 fatty acids were found that have abnormal electroretinograms (Benoken R M et al. 1973; Borre J M et al. 1989; and Wesinger H S et al. 1996) and rats fed deficient diets had decreased memory and cognitive ability. (Moriguichi T et al. 2000). Similar results have been observed in preterm human infants and in Rhesus monkeys fed DHA-deficient diets (Carlson S E et al. 1993; and Neuringer M et al. 1984).

The high amounts of DHA found in the brain and in the retina suggest a functional role in those tissues (Litman, B J et al. 2001). In non-human primates and human infants, supplemental DHA has been shown to increase visual acuity and cognitive abilities (Willats P 2002; Uauy R et al. 2003; Gil A et al. 2003). Deficiency of (n-3) polyunsaturated fatty acids (PUFA) during the developmental phase of neural tissues can result in irreversible functional abnormalities. Electroretinogram (ERG) data from humans and monkeys indicate decreased amplitudes and increased implicit times of both the a- and b-waves in response to (n-3) PUFA insufficiency (Anderson. G J et al. 1990, Jeffrey, B G et al. 2002). Reduced a- and b-wave amplitudes have also been reported in (n-3) deficient rats, however, retinal function was restored when the rats were fed (n-3) replete diets (Bourre, J M et al. 1989). Thus, long chain polyunsaturated fatty acids such as DHA are of major importance for optimizing neurological and visual development.

It has been determined that the canine retina is capable of synthesizing DHA from its 22-carbon precursor, docosapentaenoic acid (22:5 (n-3)), (DPA), that DHA is conserved in canine retinal tissue, and that DHA has a neurological function in canine retinal tissue. Bauer et al. reported the accumulation of DPA, but not DHA, in canine plasma phospholipids when the precursor alpha-linolenic acid (ALA) was fed (Bauer, J E et al. 1998). Therefore, it is likely that the canine retina, and presumably other nervous tissues, synthesize and utilize DHA in a manner similar to other mammalian species and that plasma DPA provides a starting point for such synthesis. However, it is unclear what quantities of DHA or DPA precursors are required to optimize neural development in companion animals.

Despite the knowledge regarding the benefits of DHA, and the benefits of dietary supplementation of DHA in humans and certain laboratory mammals, the benefits of DHA in the neurological development of domestic and companion animals, such as dogs and cats, remains largely unexplored. Thus, there is a need in the art to provide compositions and methods to impart the benefits of DHA and LPUFA to these types of animals, to improve their visual acuity and to provide related neurological advantages. The present invention meets this need.

### SUMMARY OF THE INVENTION

One aspect of the invention features a dietary composition comprising one or more long chain polyunsaturated fatty acids (LCPUFA), in an amount effective for improving visual acuity in an animal, preferably a companion animal such as a dog or cat. In various embodiments, the composition is a pet food composition or a dietary supplement. The LCPUFA may include at least one of arachidonic acid, linoleic acid, eicosapentaenoic acid, docosapentaenoic acid, or docosahexaenoic acid, and may be present in an amount of at least about 0.1% to about 10% by weight of the composition, more specifically between about 1.8% to about 5.0% by weight of the composition. The dietary composition may also comprise vitamins or minerals in amounts effective to promote health of the companion animal, or substances that sustain or promote ocular health or visual acuity, or at least one type of probiotic organism.

Another aspect of the invention features a method for enhancing visual acuity in an animal comprising administering to the animal one or more LCPUFA in an amount effective to enhance visual acuity in the animal. In this aspect of the invention, the LCPUFA may include one or more of arachidonic acid, linoleic acid, alpha-linolenic acid, eicosapentaenoic acid, docosapentaenoic acid, or docosahexaenoic acid. In certain embodiments, the animal is a companion animal, preferably a dog or a cat.

In one embodiment, the LCPUFA are administered to the animal during gestation. In another embodiment, the LCPUFA are administered to the animal during the period spanning parturition through about twelve weeks after parturition. In another embodiment, the LCPUFA are administered to the animal during gestation and during the period spanning parturition through about twelve weeks after parturition.

In various embodiments, the LCPUFA are administered in a pet food composition or a dietary supplement. In another embodiment, the LCPUFA are administered in milk from a lactating animal to which has been administered one or more LCPUFA. In other embodiments, the LCPUFA are administered in a pet food composition or dietary supplement and in milk from a lactating animal to which has been administered one or more LCPUFA.

The LCPUFA may be administered to the animal in various regimens. In one embodiment, the LCPUFA are administered on a daily basis. In another embodiment, the LCPUFA are administered to the animal as part of a dietary regimen. In specific embodiments, the duration of the dietary regimen ranges from parturition to about 12 weeks of age.

Other features and advantages of the invention will be understood from the drawings, detailed description and examples that follow.

### BRIEF DESCRIPTION OF THE DRAWINGS

FIG. 1. An ERG series from a puppy in the Lo/Hi group. The intensity of the stimulus increased in half log-unit steps, eliciting ERGs starting from the bottom. For all animals, the amplitudes and implicit times of both the a- and b-waves were determined at the 8^{th} (third most intense) flash intensity. Mean values are reported in Table 5.4. The ä is calculated from the top three a-wave slopes only.

FIG. 2. Mean plasma PL content of LA and AA (Panel A) and major (n-3) fatty acids (Panel B) during suckling. Because no time effects were observed for any fatty acid, data from all four sample days during suckling were pooled. Letters not in common for individual fatty acids are significantly different with respect to diet at p<0.05 (n=3 litters per diet group). Error bars indicate S.D. values.

FIG. 3. Mean plasma PL content of LA and AA (Panel A) and (n-3 fatty) acids (Panel B) after weaning. Because no time effects were observed for any fatty acid, data from both sample days were pooled. Letters not in common for individual fatty acids are significantly different with respect to diet at p<0.05 (n=3 litters per diet group). Error bars indicate S.D. values.

### DETAILED DESCRIPTION OF ILLUSTRATIVE EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The following abbreviations may be used in the specification and examples: AA, arachidonic acid; ä, increase in magnitude of the slopes of the descending limb of the a-wave; ai, a-wave implicit time; ALA, alpha-linolenic acid; a-amp, a-wave amplitude; ANOVA, analysis of variance; bi, b-wave implicit time; b-amp, b-wave amplitude; DHA, docosahexaenoic acid; DM, dry matter; DPA, docosapentaenoic acid; EPA, eicosapentaenoic acid; ERG, electroretinogram; Hi/Lo, diet containing large amount of ALA and small amounts of (n-3) LCPUFA; It, threshold intensity-light intensity at which the initial a-wave is observed; Km, Michaelis-Menton constant; LA, linoleic acid; LCPUFA, long chain polyunsaturated fatty acid; Lo/Hi, diet containing small amount of ALA and large amounts of (n-3) LCPUFA; Lo/Lo, diet containing small amount of ALA and small amounts of (n-3) LCPUFA Lo/Mod, diet containing small amount of ALA and moderate amounts of (n-3) LCPUFA; PL, phospholipids; PUFA, polyunsaturated fatty acid; ROS, rod outer-segment.

“Effective amount” refers to an amount of a compound, material, or composition, as described herein that is effective to achieve a particular biological result. Such results include, but are not limited to, enhancing visual acuity, enriching blood plasma polyunsaturated fatty acid concentration in an animal, or enriching the polyunsaturated fatty acid concenctration in the milk from a lactating anima. Such effective activity may be achieved, for example, by administering the compositions of the present invention to the animal.

The term “visual acuity” or “visual performance” are used interchangeably herein, and refer to the eye's ability to resolve fine details or small distances, as measured by any means suitable in the art, including electroretinography. “Enhanced visual acuity” refers to any improvement in the eye's ability to resolve fine details or small distances, as measured by any means suitable in the art.

As used herein, “long chain polyunsaturated fatty acids” or “LCPUFA” refers to any monocarboxylic acid having at least 20 carbon atoms and at least two double bonds. Non-limiting examples of LCPUFA include (n-6) fatty acids such as arachidonic acid, and (n-3) fatty acids such as eicosapentaenoic acid, docosapentaenoic acid and docosahexaenoic acid.

The present invention relates to any animal, preferably a mammal, and more preferably, companion animals. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Dogs and cats are most preferred, and dogs are exemplified herein.

As used herein, the term “pet food” or “pet food composition” means a composition that is intended for ingestion by an animal, and preferably by companion animals. A “complete and nutritionally balanced pet food,” is one that contains all known required nutrients in appropriate amounts and proportions based on recommendations of recognized authorities in the field of companion animal nutrition, and is therefore capable of serving as a sole source of dietary intake to maintain life or promote production, without the addition of supplemental nutritional sources. Nutritionally balanced pet food compositions are widely known and widely used in the art.

As used herein, a “dietary supplement” is a product that is intended to be ingested in addition to the normal diet of an animal.

Proper neural and retinal development of mammalian species depends on the presence of LCPUFA, especially DHA, during fetal development and the perinatal period. DHA is of particular importance in this regard because it has been demonstrated to enhance visual acuity, memory, learning, cognitive ability, and to reduce hypertension in laboratory animals. In accordance with the present invention, it has been demonstrated that long chain polyunsaturated fatty acids made available to animals pre-natally through maternal diet and post-natally through the animals' diet is effective in promoting enhanced visual performance in the animals. Enhanced visual performance is achieved when LCPUFA are administered to the animals indirectly through their mother during gestation, directly to the animals through their diet, or administered to the animals in combinations thereof.

As described in greater detail in the Examples, enriching the canine gestation/lactation diet with (n-3) LCPUFA using fish oil resulted in increases in 20:5(n-3) and 22:6(n-3) fatty acids and a decrease in 20:4(n-6) both during suckling and after weaning of puppies. Statistical analysis revealed significantly improved visual performance in the high (n-3) LCPUFA diet group. Puppies in this group demonstrated an increased rod response as measured by the amplitude and implicit time of the a-wave. A novel parameter devised in accordance with the invention was the threshold intensity, which was measured as the initial intensity at which the a-wave was detectable. Again, puppies in the high (n-3) LCPUFA diet group responded significantly sooner thereby exhibiting greater rod sensitivity, than the control group. These findings underscore the importance of preformed (n-3) LCPUFA in the diet as a means of enriching plasma and neural tissues in DHA during perinatal development, particularly for the enhancement of visual acuity in developing canines.

It is thus important to ensure that LCPUFA such as DHA are in plentiful supply in the blood of the female mammal during gestation, and are in plentiful supply in the blood of the neonatal animal through the perinatal period, and through development of the young animal. One means to accomplish this goal is through the diet of both the pregnant female and her developing newborns.

Of particular note in this regard is that dietary LCPUFA can be provided to the newborn animal through the milk of the lactating female. In humans, dietary supplementation with fishmeal or fish oil supplements results in the deposition of (n-3) fatty acids, especially DHA, into the breast milk. The DHA content of human breast milk is proportional to the DHA content of the maternal diet. This observation appears to hold true for other mammals, including non-human primates, rats, and dogs. A dose effect is observed between the DHA content of the diet and the DHA content of the milk of lactating female dogs. (Bauer J E et al. 2004 abstract). Thus, one means to provide dietary LCPUFA to neonatal and young animals, particularly during the perinatal period, is through the milk of the lactating female.

Compositions

One embodiment of the invention features compositions comprising one or more long chain polyunsaturated fatty acids (LCPUFA) in an amount effective for the enhancement of visual acuity in animals. The LCPUFA can be present in the composition as an ingredient or additive. In preferred embodiments, the composition comprises (n-3) fatty acids such as EPA, DPA and, most preferably, DHA. The compositions enrich the blood plasma with LCPUFA in animals to which the composition is administered, and enrich the milk of a lactating animal with LCPUFA in lactating animals to which the composition is administered.

In a preferred embodiment, the compositions of the invention are pet food compositions. These will advantageously include foods intended to supply necessary dietary requirements, as well as treats (e.g., biscuits) or other dietary supplements. Optionally, the pet food compositions can be a dry composition (for example, kibble), semi-moist composition, wet composition, or any mixture thereof. In another preferred embodiment, the composition is a dietary supplement, such as a gravy, drinking water, beverage, yogurt, powder, granule, paste, suspension, chew, morsel, treat, snack, pellet, pill, capsule, tablet, or any other delivery form. In a detailed embodiment, the dietary supplement can comprise a high concentration of LCPUFA or DHA such that the supplement can be administered to the animal in small amounts, or in the alternative, can be diluted before administration to an animal. The dietary supplement may require admixing with water prior to administration to the animal.

The composition may be frozen. The LCPUFA may be pre-blended with the other components of the composition to provide the beneficial amounts needed, may be coated onto a pet food composition, or may be added to the composition prior to offering it to the animal, for example, using a sprinkled powder or a mix.

The compositions of the invention comprise LCPUFA in an amount effective to enhance visual acuity in an animal to which the composition has been administered. For pet foods, the amount of (n-3) LCPUFA as a percentage of the composition is in the range of about 0.1% to about 10% in certain embodiments, up to 5% in other embodiments, and about 2.0% in specific embodiments, of the composition on a dry matter basis, although a greater percentage can be supplied. In various embodiments, the amount is about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or more of the composition on a dry matter basis. Dietary supplements may be formulated to contain several fold higher concentrations of LCPUFA, to be amenable for administration to an animal in the form of a tablet, capsule, liquid concentrated, or other similar dosage form, or to be diluted before administrations, such as by dilution in water, spraying or sprinkling onto a pet food, and other similar modes of administration.

In an alternative embodiment, the amount of LCPUFA in the composition is a function of an amount required to establish a specified concentration of LCPUFA in the blood serum of the animal. The specified concentration of LCPUFA in the blood serum is in the range of about 0.1% to about 25% of total fatty acid content in the blood serum. In another alternative embodiment, the amount of LCPUFA in the composition is a function of an amount required to establish a specified concentration of LCPUFA in the milk of the lactating animal. The specified concentration of (n-3) LCPUFA in the milk is in the range of about 0.1% to about 7.0% of total fatty acid content in the milk.

The sources of each of the LCPUFA can be any suitable source, synthetic or natural. Preferred sources of LCPUFA include, without limitation, primrose, dark green vegetables such as spinach, algae and blue-green algae such as spirulina, plant seeds and oils such as flaxseed, canola, soybean, walnut, pumpkin, safflower, sesame, wheat germ, sunflower, corn, and hemp, and fish, especially cold-water fish such as salmon, tuna, mackerel, herring, sea bass, striped bass, shark, halibut, catfish, sardines, shrimp, and clams, and their extracted oils, or the LCPUFA may be synthesized de novo according to any means suitable in the art.

The compositions of the invention can optionally comprise supplementary substances such as minerals, vitamins, salts, condiments, colorants, and preservatives. Non-limiting examples of supplementary minerals include calcium, phosphorous, potassium, sodium, iron, chloride, boron, copper, zinc, manganese, iodine, selenium and the like. Non-limiting examples of supplementary vitamins include vitamin A, various B vitamins, vitamin C, vitamin D, vitamin E, and vitamin K. Additional dietary supplements may also be included, for example, niacin, pantothenic acid, inulin, folic acid, biotin, amino acids, and the like.

The compositions of the invention can optionally comprise one or more supplementary substances that promote or sustain general ocular health, or further enhance visual acuity. Such substances can be carotenoids such as alpha- or beta-carotene, lutein, zeaxanthin, cryptoxanthin, and lycopene, flavonoids such as proanthocyanidine, rutin, quercetin, and hesperidin, herbal extracts such as Bilberry (Vaccinium myrtilis) and Curuma root, or antioxidants such as taurine and glutathione.

In various embodiments, pet food or pet treat compositions of the invention can comprise, on a dry matter basis, from about 15% to about 50% crude protein, by weight of the composition. The crude protein material may comprise vegetable proteins such as soybean, cottonseed, and peanut, or animal proteins such as casein, albumin, and meat protein. Non-limiting examples of meat protein useful herein include pork, lamb, equine, poultry, fish, and mixtures thereof.

The compositions may further comprise, on a dry matter basis, from about 5% to about 40% fat, by weight of the composition. The compositions may further comprise a source of carbohydrate. The compositions may comprise, on a dry matter basis, from about 15% to about 60% carbohydrate, by weight of the composition. Non-limiting examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

The compositions may also comprise at least one fiber source. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide additional to the short chain oligofructose, mannanoligofructose, soy fiber, arabinogalactan, galactooligosaccharide, arabinoxylan, or mixtures thereof. Alternatively, the fiber source can be a fermentable fiber. Fermentable fiber has previously been described to provide a benefit to the immune system of a companion animal. Fermentable fiber or other compositions known to those of skill in the art which provide a prebiotic composition to enhance the growth of probiotic microorganisms within the intestine may also be incorporated into the composition to aid in the enhancement of the benefit provided by the present invention to the immune system of an animal. Additionally, probiotic microorganisms, such as Lactobacillus or Bifidobacterium species, for example, may be added to the composition.

In a detailed embodiment, the composition is a complete and nutritionally balanced pet food. In this context, the pet food may be a wet food, a dry food, or a food of intermediate moisture content, as would be recognized by those skilled in the art of pet food formulation and manufacturing. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15%, and therefore is presented, for example, as small biscuit-like kibbles. The compositions and dietary supplements may be specially formulated for adult animals, or for older or young animals, for example, a “puppy chow,” “kitten chow,” or “senior” formulation. In general, specialized formulations will comprise energy and nutritional requirements appropriate for animals at different stages of development or age.

Certain aspects of the invention are preferably used in combination with a complete and balanced food (for example, as described in National Research Council, 1985, Nutritional Requirements for Dogs, National Academy Press, Washington D.C., or Association of American Feed Control Officials, Official Publication 1996). That is, compositions comprising LCPUFA, or DHA according to certain aspects of this invention are preferably used with a high-quality commercial food. As used herein, “high-quality commercial food” refers to a diet manufactured to produce the digestibility of the key nutrients of 80% or more, as set forth in, for example, the recommendations of the National Research Council above for dogs. Similar high nutrient standards would be used for other animals.

The skilled artisan will understand how to determine the appropriate amount of LCPUFA or DHA to be added to a given composition. Such factors that may be taken into account include the type of composition (e.g., pet food composition versus dietary supplement), the average consumption of specific types of compositions by different animals, and the manufacturing conditions under which the composition is prepared. Preferably, the concentrations of LCPUFA or DHA to be added to the composition are calculated on the basis of the energy and nutrient requirements of the animal. According to certain aspects of the invention, the LCPUFA or DHA can be added at any time during the manufacture and/or processing of the composition. This includes, without limitation, as part of the formulation of the pet food composition or dietary supplement, or as a coating applied to the pet food composition or dietary supplement.

The compositions can be made according to any method suitable in the art such as, for example, that described in Waltham Book of Dog and Cat Nutrition, Ed. ATB Edney, Chapter by A. Rainbird, entitled “A Balanced Diet” in pages 57 to 74, Pergamon Press Oxford.

Methods

Another aspect of the invention features methods for enhancing the visual acuity in an animal comprising administering to the animal a composition comprising one or more LCPUFA in an amount effective to enhance visual acuity in the animal. In a detailed embodiment, the composition is a pet food composition or a dietary supplement, as exemplified herein. In a further detailed embodiment, the LCPUFA is an (n-3) LCPUFA, including but not limited to, EPA, DPA and DHA. Animals may include any domesticated or companion animals as described above. In certain embodiments, the animal is a companion animal such as a dog or cat. In one embodiment, the animal is a dog.

The compositions can be administered to the animal by any of a variety of alternative routes of administration. Such routes include, without limitation, oral, intranasal, intravenous, intramuscular, intragastric, transpyloric, subcutaneous, rectal, and the like. Preferably, the compositions are administered orally. As used herein, the term “oral administration” or “orally administering” means that the animal ingests or a human is directed to feed, or does feed, the animal one or more of the inventive compositions described herein.

Wherein the human is directed to feed the composition, such direction may be that which instructs and/or informs the human that use of the composition may and/or will provide the referenced benefit, for example, the enhancement of visual acuity in the animal. Such direction may be oral direction (e.g., through oral instruction from, for example, a physician, veterinarian, or other health professional, or radio or television media (i.e., advertisement), or written direction (e.g., through written direction from, for example, a physician, veterinarian, or other health professional (e.g., prescriptions), sales professional or organization (e.g., through, for example, marketing brochures, pamphlets, or other instructive paraphernalia), written media (e.g., internet, electronic mail, or other computer-related media), and/or packaging associated with the composition (e.g., a label present on a container holding the composition).

Administration can be on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the animal or otherwise contacted with or admixed with daily feed or food. When utilized as a daily feed or food, administration will be well known to those of ordinary skill.

Administration can also be carried out as part of a diet regimen in the animal. For example, a diet regimen may comprise causing the regular ingestion by the animal of a composition comprising one or more LCPUFA, preferably DHA, in an amount effective to enhance visual acuity in the animal. Regular ingestion can be once a day, or two, three, four, or more times per day, on a daily basis. The goal of regular ingestion is to provide the animal with the preferred daily dose of LCPUFA, as exemplified herein.

According to the methods of the invention, administration of the LCPUFA, including administration as part of a diet regimen, can span a period of time ranging from gestation through the adult life of the animal. In a preferred embodiment, the duration of the administration ranges from gestation through about 36 months after parturition. In a more preferred embodiment, the duration of the administration ranges from gestation through about 30 months after parturition. In a still more preferred embodiment, the duration of the administration ranges from gestation through about 24 months after parturition. In a still more preferred embodiment, the duration of the administration ranges from gestation through about 18 months after parturition. In a still more preferred embodiment, the duration of the administration ranges from gestation through about 12 months after parturition. In a still more preferred embodiment, the duration of the administration ranges from gestation through about 40 weeks after parturition. In a still more preferred embodiment, the duration of the administration ranges from gestation through about 35 weeks after parturition. In a still more preferred embodiment, the duration of the administration ranges from gestation through about 30 weeks after parturition. In a still more preferred embodiment, the duration of the administration ranges from gestation through about 25 weeks after parturition. In a still more preferred embodiment, the duration of the administration ranges from gestation through about 20 weeks after parturition. In a still more preferred embodiment, the duration of the administration ranges from gestation through about 18 weeks after parturition. In a still more preferred embodiment, the duration of the administration ranges from gestation through about 16 weeks after parturition. In a still more preferred embodiment, the duration of the administration ranges from gestation through about 14 weeks after parturition. In a still more preferred embodiment, the duration of the administration ranges from gestation through about 12 weeks after parturition. In an alternative embodiment, the duration of the administration ranges from gestation through about 10 weeks after parturition. In another alternative embodiment, the duration of the administration ranges from gestation through about 8 weeks after parturition. In another alternative embodiment, the duration of the administration ranges from gestation through about 6 weeks after parturition.

In an alternative embodiment, the inventive method comprises administering to the animal milk from a lactating animal to which has been administered one or more LCPUFA. Milk may be enriched with LCPUFA by administering to a lactating animal one or more LCPUFA in an amount sufficient to establish a specified concentration of said LCPUFA in the milk of the lactating animal. The LCPUFA-enriched milk can be administered to an animal in order to enhance the visual acuity in that animal.

The LCPUFA can be administered to the lactating animal according to the inventive methods described herein. In a preferred embodiment, the lactating animal is administered a composition comprising one or more LCPUFA. In a more preferred embodiment, the lactating animal is administered a composition comprising DHA. The composition comprising one or more LCPUFA that is administered to the lactating animal can be a pet food composition or dietary supplement, as exemplified herein. The composition may be administered to the lactating animal before conception, during gestation, and after parturition during the suckling period. The lactating animal may be the parent of the animal to which the milk is administered. The milk may be administered via suckling, or may be administered after isolation from the lactating animal. The milk can be administered on an as-needed or as-desired basis, or as part of a diet regimen, as described herein.

In another alternative embodiment, the inventive method comprises administering LCPUFA to the animal during gestation, by passage from the mother animal to which has been administered one or more LCPUFA. In a preferred embodiment, the mother animal is administered a composition comprising one or more LCPUFA. In a more preferred embodiment, the mother animal is administered a composition comprising DHA. The composition comprising one or more LCPUFA that is administered to the mother animal can be a pet food composition or dietary supplement, as exemplified herein. The composition may be administered to the mother animal from before the time of estrus through parturition.

In still another alternative embodiment, the LCPUFA is administered to the animal both during gestation and after parturition according to the details set forth above.

The amount of composition utilized in the various embodiments of the methods of the invention may be dependent on a variety of factors, including the health, condition, and/or age of the animal, the quality of the pet food composition or dietary supplement, and species, size or breed of the animal.

Determination of the improvement of visual performance of the animals achieved by practicing the methods of the invention may be determined by any means suitable in the art. One particularly effective means to measure visual acuity is electroretinography (ERG).

Electroretinography is a sensitive and quantitative measure of retinal function in humans and animals (Armington, JC 1974, Gouras, P 1970). The ERG recording represents photoreceptor responses, and their subsequent post-synaptic signals, to a series of varying-intensity flash stimuli. It is a summation of responses across the retina and includes the responses of many retinal cell types. Major ERG components have been studied in dogs (Yanese, J et al. 1996), and studies support using dogs as a suitable model for studies of human retinal physiology and pathology (Kommonen, B et al. 1991). Parameters to assess using ERG include, without limitation, a-wave amplitude, b-wave amplitude, a-wave implicit times, b-wave implicit times, and ä, which is derived from the slope of the a-wave.

The following examples are provided to describe the invention in greater detail. The examples are intended illustrate, not to limit, the invention.

### EXAMPLE 1

### Animals and Diets

An existing breeding colony of dogs provided bred hound/Labrador retriever dogs and their puppies for this study, as previously described (Bauer, J E et al. 2004). Dogs were individually maintained in kennels according to the American Physiological Society Guidelines for Animal Research. Twelve clinically normal, sexually intact female dogs from this colony, ages 2 to 4 years, were randomly assigned to one of four diet groups (3 dogs per diet group). The diets were fed from the time of estrus, breeding, artificial insemination, and throughout gestation, parturition, and lactation. The numbers of puppies available for study in each group were variable due to litter size differences obtained from each breeding.

The dry, extruded-type diets were complete and balanced and prepared by Nestle-Purina PetCare Company, St. Louis, Mo. The diet ingredients, by weight, included Brewers milled rice, 37.7%, dehulled soybean meal 33.8%, poultry by-product meal, 4.8%, Supro 620® protein, 3.7%, dicalcium phosphate, 2.1% pea fiber, 1.9% flavor coating, 1.0%, potassium chloride, 0.5%, taurine 0.5%, sodium chloride, 0.5%, vitamin pre-mix, 0.3% and mineral pre-mix, 0.3%, and choline chloride, 0.1%. The vitamin premix, contained 146,320 mg/kg nicotinic acid, 10,350 mg/kg vitamin A acetate, 90,000 mg/kg dl-α-tocopherol acetate, 84,000 μg/kg cholecalciferol, 52,000 mg/kg thiamine mononitrate, 51,060 mg/kg calcium D-pantothenate, 24,400 mg/kg riboflavin, 14,520 mg/kg pyridoxine hydrochloride, 6,000 mg/kg folic acid, 508 mg/kg menadione sodium bisulfite, 93 mg/kg vitamin B-12, and 36.8 mg/kg biotin. The mineral mix contained 65 g/kg zinc as zinc sulfate, 39 g/kg iron as ferrous sulfate, 18.25 g/kg manganese as manganese sulfate, 3.2 g/kg copper as copper sulfate 655 mg/kg iodine as calcium iodate, and 50 mg/kg selenium as selenium selenite. The remaining 13.3% was provided by various dietary oils which provided the desired fatty acid profiles.

All diets contained sufficient amounts of LA ranging from 1.8-3.5% dry matter (DM), and differed in their fatty acid compositions. Each diet consisted of approximately 15% total fat and contained one of the following as its primary fat source: beef tallow, linseed oil, “low” amounts of Menhaden fish oil or “high” amounts of Menhaden fish oil. Each diet was designed to have a unique ratio of its ALA to (n-3) LCPUFA content. Based on these relative amounts, the diets were designated as low ALA/low (n-3) LCPUFA (Lo/Lo, tallow); high ALA/low (n-3) LCPUFA (Hi/Lo, linseed oil); low ALA/moderate (n-3) LCPUFA (Lo/Mod, low Menhaden fish oil); and low ALA/high (n-3) LCPUFA (Lo/Hi, high Menhaden fish oil) (Table 1). The expected nutrient composition of the diets (by weight) was 71.8% moisture, 27.8% protein, 15.1% fat, 9.1% moisture, 5.7% ash, 2.0% crude fiber and the remainder carbohydrate. The diets were analyzed by Purina Laboratories (St. Louis, Mo.) and shown to be within expected analytical variance of these targets (Table 2).

TABLE 1
Experimental diet fatty acid concentration (% dry matter basis)*

Diet

| Fatty Acid | Lo/Lo | Lo/Mod | Lo/Hi | Hi/Lo |
|---|---|---|---|---|
| Saturated | 6.02 | 5.46 | 6.95 | 2.55 |
| Monounsaturated | 6.55 | 6.14 | 6.17 | 4.22 |
| 18:2n-6 | 1.75 | 2.61 | 1.16 | 3.5 |
| 20:4n-6 | 0.02 | 0.03 | 0.05 | 0.02 |
| 18:3n-3 | 0.14 | 0.29 | 0.2 | 6.82 |
| 20:5n-3 | 0.02 | 0.19 | 0.5 | 0.02 |
| 22:5n-3 | 0.02 | 0.05 | 0.11 | 0.02 |
| 22:6n-3 | 0.02 | 0.19 | 0.55 | 0.02 |
| % total fatty acids | 14.54 | 14.96 | 15.69 | 17.17 |
| % total dietary fat | 15.33 | 15.90 | 16.10 | 17.47 |

*The diets are designated based on their ALA/(n-3) LCPUFA contents. For details see text.

TABLE 2
Experimental diet nutrient profiles (g/kg dry matter))*

Diet

| Fatty Acid | Lo/Lo | Lo/Mod | Lo/Hi | Hi/Lo |
|---|---|---|---|---|
| Crude protein | 307 | 304 | 299 | 314 |
| Fat | 153 | 158 | 161 | 175 |
| Nitrogen-free extract | 478 | 475 | 479 | 446 |
| Ash | 62 | 62 | 61 | 65 |
| Energy (kcal/g) | 51 | 51 | 52 | 50 |

*For ingredients of the diets see text.

After parturition, puppies were allowed to suckle ad libitum. Milk samples were collected from all mothers by manual expression during lactation on days 4, 10, 16 and 28 for lipid extraction and fatty acid profile analyses, as described previously (Bauer, J E et al. 2004). At 21 days postpartum, a gruel consisting of the mothers' respective diets and water was offered to the puppies three times a day in addition to suckling. Gradually, the time the puppies spent suckling was decreased until they were completely weaned by day 42. Upon weaning, puppies were continued on the same diets as their mothers until 12 weeks of age.

The puppies were weighed daily until 6 weeks of age, and twice per week thereafter to monitor proper growth and development. If any failure-to-thrive issues arose, the puppy was removed from the study and supplemented to ensure proper nutrition. Generally this occurred only when litter size was large; small puppies were the most likely to be removed.

### EXAMPLE 2

### Plasma Lipid Extraction and Electroretinography

EDTA blood samples were taken from the puppies on day 4, 10, 16, 28, 70 and 84 of age. Food was withheld overnight on day 70 and 84. The puppies were removed from their mothers three hours prior to blood collection. Two milliliters of blood were taken on each sample day through day 16. On day 28, 4 mL were collected, and on days 70 and 84, 7 mL were obtained. Plasma and milk total lipids were extracted using chloroform:methanol (2: 1, v/v) via a modified Folch procedure (Folch, J et al. 1957). Total plasma phospholipids (PL) were separated via thin layer chromatography (Bauer, J E et al. 1997). Fatty acid methyl esters were prepared of the plasma PL and milk total lipids and fatty acid profiles were determined via capillary gas chromatography (Bauer, J E et al. 1998).

At 12 weeks of age, retinal function of the puppies was assessed via electroretinography (ERG). Electroretinograms of both eyes of all puppies studied were recorded using a computer-based ERG acquisition system designed using Windows-based software (Microsoft corp., Redmond, Wash.).

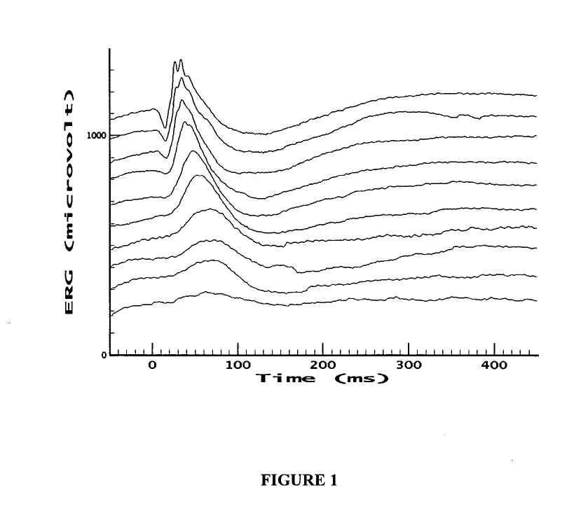

Each eye was tested separately using a series of square-wave flash stimuli, 50 msec in duration, with an inter-flash interval of 5 seconds from a white-light emitting diode (LED) placed approximately 1 cm from the cornea. The ERGs were obtained at 10 increasing 0.5 log unit intensity steps up to b-wave saturation. The highest intensity setting has been found to saturate the rod response in canine. The parameters used to assess ERG characteristics were a- and b-wave amplitude and a- and b-wave implicit times. A typical ERG series obtained with the equipment used in this study is shown in FIG. 1.

An additional parameter, ä, was derived from the slope of the a-wave (Diau, G Y et al. 2003). The ERG software used in the study calculated the slopes of the descending limb of the a-wave for the three highest intensity responses; the increase in magnitude of these slopes as a function of intensity is reported as the parameter ä. The slope for each of the three intensities was then plotted against flash intensity. The resultant data were modeled with linear regression to yield a as the calculated slope parameter (Breton, M E et al. 1994). The slope ä represents the increase in initial response with increasing light intensity (Li, Z et al. 2000).

The effects of diet and time on individual plasma PL fatty acid data were evaluated via repeated measures ANOVA using “litter” as the experimental unit. For the milk data “dam” was the experimental unit (n=3). Where appropriate, multiple comparisons for main effects of diet, time, and diet*time interactions were performed at p<0.05 (Statistix 7.0; Analytical Software, Tallahassee, Fla.). Where there was a significant interaction of group and time, contrasts were made for each plasma PL fatty acid using Bonferroni's test to determine where the difference occurred. An experiment-wide type I error of 0.05 was maintained. Statistical analyses were performed on ERG parameters using data obtained from the 8^{th} flash intensity via one-way ANOVA with Bonferroni comparisons performed at p<0.05. Because each eye was tested separately, sample sizes of each group used for statistical analyses was twice that of the number of puppies in each group.

### EXAMPLE 3

### Effect of Dietary Fatty Acids on Plasma Lipid Concentration During Suckling Period

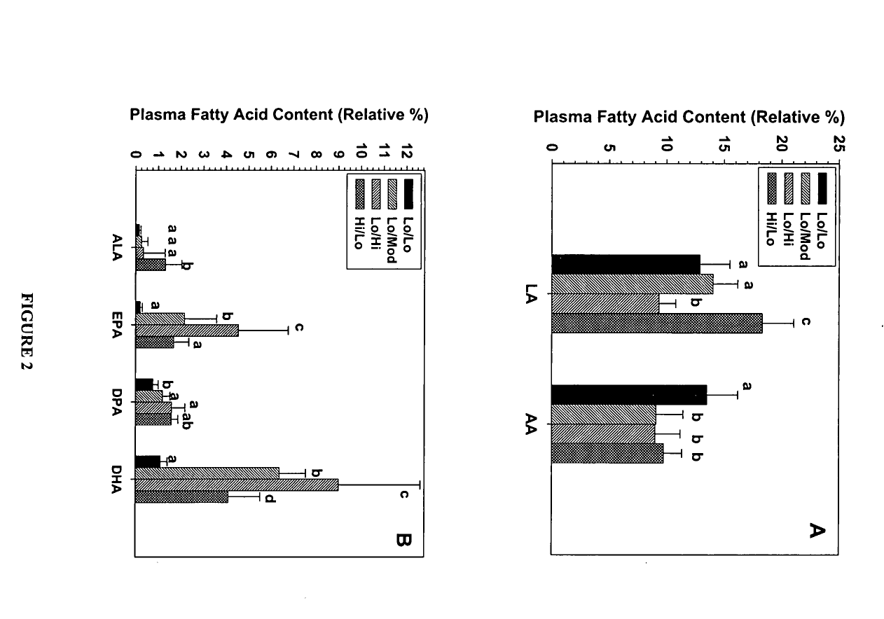

A dose response of dietary LA (from milk) was observed in neonatal plasma (FIG. 2, Panel A). Milk from the Hi/Lo group contained the highest concentration of LA (Table 3); consequently so did plasma PL from neonates in this group. Plasma PL arachidonate in neonates from the Lo/Lo group, was significantly higher than all other groups (FIG. 2, Panel A). A summary of the mean plasma PL content of major PUFA during suckling is presented in Table 4. Values presented are mean±S.D for each sample day during the suckling period.

TABLE 3
Mean ± S.D. dietary content (in milk) of major (n-6)
and (n-3) polyunsaturated fatty acids during suckling

| | FATTY ACID | | | | | |
|---|---|---|---|---|---|---|
| DIET | 18:2 n-6 | 20:4 n-6 | 18:3 n-3 | 20:5 n-3 | 22:5 n-3 | 22:6 n-3 |
| Lo/Lo | 7.27 ± 1.63^{a} | 0.81 ± 0.35 | 0.63 ± 0.40^{a} | 0.53 ± 0.44^{a} | 0.63 ± 0.52^{ab} | 0.30 ± 0.25^{a} |
| Lo/Mod | 10.5 ± 2.23^{b} | 0.70 ± 0.22 | 1.42 ± 1.20^{a} | 1.18 ± 0.24^{b} | 0.88 ± 0.40^{ab} | 1.41 ± 0.69^{b} |
| Lo/Hi | 6.73 ± 2.20^{a} | 0.74 ± 0.34 | 1.54 ± 1.62^{a} | 1.87 ± 0.53^{c} | 1.00 ± 0.24^{a} | 2.28 ± 0.88^{c} |
| Hi/Lo | 14.8 ± 1.58^{c} | 0.73 ± 0.17 | 22.3 ± 7.53^{b} | 0.59 ± 0.21^{a} | 0.35 ± 0.37^{b} | 0.19 ± 0.34^{a} |

Letters not in common for individual fatty acids are significantly different at p < 0.05 (n = 3 dogs per group). Mean milk fat concentration was 8.0 ± 2.0% on an as-is basis.

TABLE 4
Relative percent (mean ± S.D.) of major (n-6) and (n-3) polyunsaturated
fatty acids in neonatal plasma PL during suckling

| | | FATTY ACID | | | | | |
|---|---|---|---|---|---|---|---|
| DIET | DAY | 18:2 n-6 | 20:4 n-6 | 18:3 n-3 | 20:5 n-3 | 22:5 n-3 | 22:6 n-3 |
| Lo/Lo | 4 | 13.2 ± 0.5 | 15.0 ± 2.7 | 0.2 ± 0.1 | 0.1 ± 0.1 | 0.6 ± 0.1 | 1.0 ± 0.4 |
| | 10 | 14.1 ± 2.7 | 13.9 ± 2.4 | 0.2 ± 0.1 | 0.2 ± 0.0 | 0.8 ± 0.0 | 1.1 ± 0.3 |
| | 16 | 15.2 ± 0.9 | 13.6 ± 1.5 | 0.2 ± 0.1 | 0.2 ± 0.0 | 0.9 ± 0.4 | 1.2 ± 0.1 |
| | 28 | 9.4 ± 1.0 | 11.7 ± 3.9 | 0.1 ± 0.0 | 0.3 ± 0.1 | 0.7 ± 0.2 | 1.2 ± 0.4 |
| Hi/Lo | 4 | 18.2 ± 0.9 | 11.2 ± 1.9 | 0.87 ± 0.4 | 1.1 ± 0.6 | 1.4 ± 0.4 | 2.7 ± 0.2 |
| | 10 | 20.0 ± 2.4 | 9.1 ± 1.2 | 1.4 ± 0.1 | 1.6 ± 0.7 | 1.5 ± 0.1 | 3.8 ± 0.1 |
| | 16 | 18.1 ± 4.0 | 8.9 ± 1.4 | 1.1 ± 0.6 | 1.8 ± 0.8 | 1.8 ± 0.3 | 4.3 ± 0.7 |
| | 28 | 16.5 ± 3.1 | 9.8 ± 1.4 | 1.9 ± 1.2 | 2.2 ± 0.3 | 1.7 ± 0.3 | 5.6 ± 2.0 |
| Lo/Mod | 4 | 13.3 ± 2.5 | 10.8 ± 1.0 | 0.3 ± 0.1 | 1.3 ± 0.5 | 1.3 ± 0.3 | 7.2 ± 0.6 |
| | 10 | 12.4 ± 2.7 | 8.1 ± 1.7 | 0.6 ± 0.4 | 1.5 ± 0.3 | 1.0 ± 0.3 | 5.9 ± 1.1 |
| | 16 | 15.5 ± 0.6 | 8.7 ± 4.1 | 0.04 ± 0.08 | 3.3 ± 2.4 | 1.3 ± 0.5 | 6.7 ± 1.4 |
| | 28 | 14.8 ± 1.6 | 8.8 ± 1.8 | 0.09 ± 0.08 | 2.5 ± 1.1 | 1.2 ± 0.5 | 5.7 ± 1.2 |
| Lo/Hi | 4 | 9.2 ± 2.1 | 8.5 ± 0.8 | 0.03 ± 0.05 | 2.0 ± 1.2 | 1.2 ± 0.2 | 5.2 ± 1.9 |
| | 10 | 10.3 ± 0.05 | 9.6 ± 1.4 | 0.1 ± 0.2 | 5.4 ± 1.8 | 1.5 ± 0.5 | 9.3 ± 2.9 |
| | 16 | 9.3 ± 2.2 | 10.1 ± 4.4 | 1.2 ± 2.0 | 4.1 ± 0.7 | 2.2 ± 1.3 | 10.3 ± 3.5 |
| | 28 | 8.6 ± 0.2 | 7.8 ± 0.2 | 0.1 ± 0.2 | 6.7 ± 1.9 | 1.7 ± 0.3 | 11.2 ± 3.9 |
| ANOVA | time | 0.0597 | 0.2850 | 0.7250 | 0.0024 | 0.0537 | 0.0663 |
| (p) | diet | 0.0000 | 0.0001 | 0.0002 | 0.0000 | 0.0000 | 0.0000 |

No time effects within diet groups were observed at p < 0.05 (n = 3 litters per diet group). Dietary differences for individual fatty acids are indicated in FIG. 2.

With respect to the plasma (n-3) fatty acids, dose responses were observed for ALA, EPA and DHA. Puppies in the Hi/Lo group received the largest relative amount of ALA in their diets (22.3±7.5% S.D.) and their plasma PL also contained the highest concentration of ALA (1.3±0.7% S.D.). These values were significantly different from all other diets at p<0.05. Neither dietary nor neonatal plasma ALA were different among the remaining dietary groups (FIG. 2, Panel B).

Milk EPA and DHA were highest in the two fish-oil diet groups (Lo/Mod and Lo/Hi). Likewise neonatal plasma from these groups contained the largest amounts of EPA and DHA in the PL fraction (FIG. 2, Panel B). Neonatal plasma PL from the Lo/Hi group contained 4.6±2.2% S.D. EPA compared to 2.2±1.4% S.D. in the Lo/Mod group. These values were statistically significantly different from each other as were the concentrations of dietary (milk) EPA in these two groups (p<0.05).

Despite a two-fold, and statistically significant, difference between milk EPA in the Lo/Mod and Hi/Lo groups, mean EPA in plasma PL from neonates in these groups were not significantly different. However, milk ALA differed considerably between these two diet groups. Mean plasma PL EPA from puppies in the Hi/Lo group was 1.7±0.7% S.D. (FIG. 2, Panel B).

Puppy dietary (milk) DPA was highest in the two fish-oil groups and lowest in the Hi/Lo group (Table 3). However, DPA concentrations in plasma PL were similar between the Hi/Lo and Lo/Mod groups, with values of 1.6±0.3% S.D. and 1.2±0.3% S.D., respectively. Puppies in the Lo/Hi group had the greatest plasma PL DPA content, 1.59±0.59%, while the Lo/Lo group had the lowest, 0.77±0.23%. None of these differences attained statistical significance (FIG. 2, Panel B).

A dose response of DHA was also observed in the plasma of puppies fed diets containing (n-3) LCPUFA. DHA concentrations in plasma PL were 9.0±3.6% S.D., 6.4±1.2% S.D. and 1.1±0.3% S.D, respectively, in the Lo/Hi, Lo/Mod and Lo/Lo groups. Puppies in the Hi/Lo group received the lowest relative amount of DHA in milk, yet their plasma PL contained relatively large amounts, 4.1±1.4% S.D., of this fatty acid. Plasma DHA concentrations in all diet groups were significantly different from each other at p<0.05.

### EXAMPLE 4

### Effect of Dietary Fatty Acids on Plasma Lipid Concentration During the Post-Weaning Period

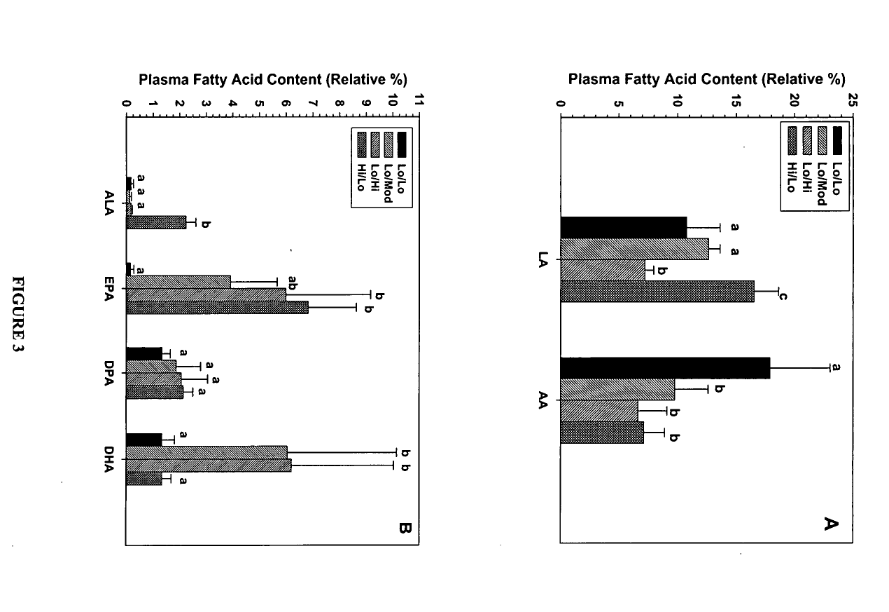

Plasma PL fatty acid profiles of the puppies consuming the dry diets were similar to those obtained during suckling. A dose response was again observed for LA (FIG. 3, Panel A). Mean plasma PL AA in the Lo/Lo group was significantly higher than all other groups during both the suckling and post-weaning periods. A summary of the mean plasma PL content of major fatty acids after weaning is presented in Table 5. Values presented are mean values for each sample day during the post-weaning period.

TABLE 5
Relative percent (mean ± S.D.) of major (n-6) and
(n-3) fatty acids in neonatal plasma PL after weaning.

| | | FATTY ACID | | | | | |
|---|---|---|---|---|---|---|---|
| DIET | DAY | 18:2 n-6 | 20:4 n-6 | 18:3 n-3 | 20:5 n-3 | 22:5 n-3 | 22:6 n-3 |
| Lo/Lo | 70 | 11.0 ± 2.3 | 18.9 ± 4.0 | 0.2 ± 0.1 | 0.3 ± 0.0 | 1.3 ± 0.4 | 1.5 ± 0.4 |
| | 84 | 10.5 ± 3.8 | 16.9 ± 6.9 | 0.2 ± 0.1 | 0.6 ± 0.1 | 1.4 ± 0.2 | 1.2 ± 0.6 |
| Hi/Lo | 70 | 17.0 ± 2.3 | 7.2 ± 1.9 | 2.3 ± 0.6 | 6.5 ± 1.7 | 2.1 ± 0.1 | 1.5 ± 0.4 |
| | 84 | 16.1 ± 2.3 | 7.1 ± 2.1 | 2.2 ± 0.2 | 7.1 ± 2.3 | 2.2 ± 0.5 | 1.2 ± 0.3 |
| Lo/Mod | 70 | 11.8 ± 0.6 | 8.3 ± 2.7 | 0.2 ± 0.02 | 3.5 ± 1.7 | 1.6 ± 0.7 | 4.7 ± 2.5 |
| | 84 | 13.4 ± 0.2 | 11.1 ± 2.7 | 0.1 ± 0.1 | 4.4 ± 2.1 | 2.1 ± 1.2 | 7.4 ± 5.5 |
| Lo/Hi | 70 | 7.9 ± 0.2 | 6.9 ± 2.8 | 0.2 ± 0.05 | 5.7 ± 3.1 | 2.1 ± 1.1 | 5.0 ± 2.5 |
| | 84 | 6.6 ± 0.3 | 6.4 ± 2.6 | 0.1 ± 0.02 | 6.3 ± 4.0 | 2.0 ± 1.1 | 7.4 ± 5.1 |
| ANOVA | time | 0.7391 | 0.9623 | 0.5636 | 0.6279 | 0.6021 | 0.3007 |
| (p) | diet | 0.0000 | 0.0000 | 0.0000 | 0.0004 | 0.3245 | 0.0032 |

No time effects within diet groups were detected at p < 0.05 (n = 3 litters per diet group). Dietary differences for individual fatty acids are indicated in FIG. 3.

The Hi/Lo group again showed significantly greater ALA, 2.2±0.4% S.D., in plasma PL (FIG. 3, Panel B). The other three groups, whose plasma PL ALA was on the order of 0.15% of total fatty acids, were not significantly different from each other.

The Hi/Lo group also had the largest concentrations of EPA and DPA in the plasma PL fraction (FIG. 3, Panel B). Mean EPA during the post-weaning period was 6.8 ±1.8% S.D. in the Hi/Lo group, compared to 3.9±1.8% S.D. and 6.0±3.2% S.D., respectively, in the Lo/Mod and Lo/Hi diet groups. These values, however, were not statistically different from each other. Plasma PL fatty acids from puppies in the Lo/Lo diet group contained considerably less EPA, 0.2±0.1% S.D., yet due to considerable variability in individual responses, this difference was not significantly different from puppies in the Lo/Mod group. In addition, statistical differences in plasma PL DPA concentrations were not observed amongst diets at p<0.05.

During the post-weaning period, a dose response of fish oil was not as apparent in the plasma of the Lo/Lo, Lo/Mod and Lo/Hi puppies. This observation is in contrast to data from the suckling period in which a dose effect of DHA was clearly observed in the plasma of neonates from these diet groups. Plasma PL DHA content in the fish-oil groups were significantly greater than the other two groups, but were not significantly different from each other due to sample variability. Mean plasma PL DHA concentrations during the post-weaning period were 6.2±3.8% S.D. and 6.1±4.1% S.D., respectively, in the Lo/Hi and Lo/Mod groups and were not statistically different (FIG. 3, Panel B). These values compared with 1.3±0.4% and 1.3±0.5% S.D., respectively in the Hi/Lo and Lo/Lo diet groups and both were statistically different from the fish oil-containing groups (p<0.05).

### EXAMPLE 5

### Effect of Dietary Fatty Acids on Retinal Function

Retinal function was assessed at 12 weeks of age via flash electroretinography (FIG. 1). Parameters of interest were the a- and b-wave amplitudes (a-amp and b-amp, respectively), the implicit times of the a- and b-waves (aᵢ and bᵢ, respectively), and the derived parameter, ä. Statistical analyses were performed on data obtained at the eighth light intensity because the highest intensity (10^{th}) is known to saturate the rod response in canines. A summary of these parameters, including sample sizes, is presented in Table 6.

TABLE 6
Mean ± S.D. values for ERG parameters obtained
at 8^{th} light intensity (a-amp, b-amp, aᵢ and bᵢ)
threshold intensity (Iₜ).

| DIET | a-amp(μV) | b-amp (μV) | aᵢ (ms) | bᵢ (ms) | ä | Iₜ |
|---|---|---|---|---|---|---|
| Lo/Lo | 31.6 ± 19.6^{a} | 172.3 ± 60.0^{ab} | 6.1 ± 2.3^{a} | 35.5 ± 4.1^{a} | 1.8 ± 1.0^{a} | 6.2 ± 1.0^{a} |
| n = 36 | | | | | | |
| Lo/Mod | 24.6 ± 8.8^{a} | 153.2 ± 49.5^{b} | 5.6 ± 1.5^{ab} | 34.9 ± 2.9^{a} | 1.6 ± 0.5^{a} | 5.8 ± 0.8^{ab} |
| n = 26 | | | | | | |
| Lo/Hi | 49.5 ± 16.3^{b} | 197.8 ± 47.5^{a} | 4.4 ± 1.2^{b} | 33.0 ± 4.3^{ab} | 2.5 ± 1.0^{b} | 5.3 ± 0.6^{b} |
| n = 20 | | | | | | |
| Hi/Lo | 43.5 ± 18.4^{b} | 169.7 ± 42.6^{ab} | 5.0 ± 1.4^{ab} | 32.1 ± 3.2^{b} | 1.9 ± 0.7^{ab} | 5.9 ± 0.8^{a} |
| n = 30 | | | | | | |
| p-value | 0.0000 | 0.0350 | 0.0026 | 0.0013 | 0.0066 | 0.0017 |
| (ANOVA) | | | | | | |

For abbreviations see text.
Letters not in common for a given parameter are significantly different at p < 0.05.

The puppies in the Lo/Hi group demonstrated the greatest response in their a-waves. Mean a-amp in this group was 49.5±16.3 μV S.D. This value was not significantly different from the mean a-amp of the Hi/Lo group, which was 43.5±18.4 μV S.D. Both groups, however, were significantly different from the Lo/Mod and Lo/Lo groups, which had mean a-amp of 24.6±8.8 μV S.D. and 31.6±19.6 μV S.D., respectively.

Mean aᵢ was lowest in the Lo/Hi group and highest in the Lo/Lo group. Implicit times were not significantly different among the Lo/Mod, Lo/Hi and Hi/Lo diet groups, whose mean aᵢ were 5.6±1.5 ms S.D., 4.4±1.2 ms S.D., and 5.0±1.4 ms S.D., respectively. The aᵢ of the Lo/Lo group was 6.1±2.3 ms S.D., which was not significantly different (p<0.05) from values obtained in the Lo/Mod or Hi/Lo groups.

The a parameter as described by Breton et al., is a measure of the time-course of activation of the phosphodiesterase cascade in the rod outer segments (ROS) (Breton, M E et al. 1994). Because the calculation of ä is based on the a-amp, the results obtained follow a similar pattern as those obtained for the a-amp. Again, visual performance in the Lo/Hi group was significantly greater than the Lo/Lo and Lo/Mod groups. Values of ä for these groups were 2.5±1.0, 1.8±1.0, and 1.6±1.5, respectively. The value of ä in the Hi/Lo group was 1.9±0.7, which was not significantly different from any other dietary group.

Dietary content of LCPUFA did not appear to have a direct effect on either of the b-wave parameters. The greatest b-amp, 197.8±47.5 μV, was observed in the Lo/Hi group, followed by the Lo/Lo, Hi/Lo and Lo/Mod groups, respectively. On average, puppies in the Hi/Lo group elicited the quickest b-wave response (lowest mean bᵢ), at 32.1±3.2 ms, which was significantly different from the Lo/Lo and Lo/Mod groups. The mean bᵢ of puppies in the Lo/Hi group was 33.0±4.3 ms, while the mean bᵢ of the Lo/Lo and Lo/Mod groups was 35.5±4.1 ms and 34.9±2.9 ms, respectively. No significant differences were detected among these latter three diet groups at p<0.05.

Not all animals responded equally to all intensities of light. Some puppies did not elicit an a-wave response until the 7^{th} or 8^{th} flash intensity, whereas others responded as early as the 4^{th} flash intensity. Based on this observation, mean values were obtained for the threshold intensity (Iₜ), which was the intensity at which the a-wave was first detected. To our knowledge, such a parameter has not been reported previously. On average, the Lo/Hi group responded to flashes at lower intensities than did the other three groups. The Lo/Mod and Lo/Hi diet groups were not significantly different from each other, but the Lo/Hi group was significantly different from the Lo/Lo and Hi/Lo groups (Table 6).

A novel parameter devised in this study is the ERG threshold intensity, the light intensity at which the initial a-wave was observed. Puppies that consumed the highest amount of (n-3) LCPUFA (Lo/Hi diet) elicited the earliest photoreceptor response, which was at a statistically significantly lower intensity than those of the Lo/Lo and Hi/Lo groups, but not different from the Lo/Mod group. These data indicate that puppies whose diets contained more (n-3) LCPUFA had lower rod thresholds (i.e. greater rod sensitivity) than puppies in the other groups. This is believed to be a consequence of the higher dietary concentrations of (n-3) LCPUFA in these two groups.

Taken together, the data presented herein indicate an advantage of dietary DHA on retinal function in young canines. Puppies consuming the highest concentrations of DHA in both milk and dry diet consistently demonstrated the greatest rod sensitivity (as measured by a-amp, aᵢ, and It) and elicited the greatest increase in the amplification of the phosphodiesterase cascade. Although visual performance in puppies fed the high ALA diet was not significantly lower than those fed DHA, it was not generally equivalent to the level of retinal function observed in the DHA-fed puppies.

### REFERENCES

1. Anderson, G. J., Connor, W. E., and Corliss, J. D. (1990) Docosahexaenoic Acid is the Preferred Dietary n-3 Fatty Acid for the Development of the Brain and Retina, Pediatr Res 27, 89-97.
2. Armington, J. C. (1974) The Electroretinogram, Academic Press, New York.
3. Bauer, J. E., McAlister, K. G., Rawlings, J. M., and Markwell, P. (1997) Molecular species of cholesteryl esters formed via plasma lecithin: cholesterol acyltransferase in fish oil supplemented dogs. Nutr. Res. 17, 861-872.
4. Bauer, J. E., Dunbar, B. L., and Bigley, K. E. (1998) Dietary Flaxseed in Dogs Results in Differential Transport and Metabolism of n-3 Polyunsaturated Fatty Acids, J Nutr 128(Suppl), 2641S-2644S.
5. Bauer, J. E., Heinemann, K. M., Bigley, K. E., Lees, G. E., and Waldron, M. K. (2004) Maternal Diet Alpha-Linolenic Acid During Gestation and Lactation Does Not Increase Canine Milk Docosahexaenoic Acid, J Nutr 134, 2035S-2038S.
6. Bauer, J. E., Heinemann, K. M., Bigley, K. E., and Waldron, M. K. (2004) Enrichment of canine milk with n-3 LCPUFA is dose-dependent on gestation/lactation diet. ABSTRACT 6^{th} Congress of ISSFAL, Brighton, UK
7. Benolken, R. M., Anderson, R. E., and Wheeler, T. G. (1973) Membrane fatty acids associated with the electrical response in visual excitation, Science. 182, 1253-54.
8. Bourre, J. M., Francois, M., Youyou, A., Dumont, O., Piciotti, M., Pascal, G., and Durand, G. (1989) The Effects of Dietary α-Linolenic Acid on the Composition of Nerve Membranes, Enzymatic Activity, Amplitude of Electrophysiological Parameters, Resistance to Poisons and Performance of Learning Tasks in Rats, J Nutr 119, 1880-1892.
9. Breton, M. E., Schueller, A. W., Lamb, T. D., and Pugh, E. N. (1994) Analysis of ERG a-Wave Amplification and Kinetics in Terms of the G-Protein Cascade of Phototransduction, Invest Ophthalmol Vis Sci 35, 295-309.
10. Carlson, S. E., Werkman, S. H., Rhodes, P. G., and Tolley, E. A. (1993) Visual-acuity development in healthy preterm infants: Effect of marine-oil supplementation, Am. J. Clin. Nutr. 58, 35-42
11. Diau, G. Y., Loew, E. R., Wijendran, V., Sarkadi-Nagy, E., Nathanielsz, P. W., and Brenna, J. T. (2003) Docosahexaenoic and Aracidonic Acid Influence on Preterm Baboon Retinal Composition and Function, Invest Ophthalmol Vis Sci 44, 4559-4566.
12. Folch, J., Lees, M., and Stanley, G. H. (1957) A Simple Method for the Isolation and Purification of Total Lipids From Animal Tissues, J Biol Chem 226, 497-509.
13. Gil, A., Ramirez, M., and Gil, M. (2003) Role of long-chain polyunsaturated fatty acids in infant nutrition, Eur. J. Clin. Nutr. 57, S31-57.
14. Gouras, P. (1970) Electroretinography: Some Basic Principles, Invest Ophthal 9, 557-569.
15. Green, P., and Yavin, E. (1996) Fatty acid composition of late embryonic and early postnatal rat brain. Lipids 31, S859-865.
16. Greiner, R. C., Winter, J., Nathanielsz, P. W., and Brenna, J. T. (1997) Brain Docosahexaenoate Accretion in Fetal Baboons: Bioequivalence of Dietary Alpha-Linolenic and Docosahexaenoic Acids, Pediatr Res 42, 826-834.
17. Jeffrey, B. G., Mitchell, D. C., Gibson, R. A., and Neuringer, M. (2002) n-3 Fatty Acid Deficiency Alters Recovery of the Rod Photoresponse in Rhesus Monkeys, Invest Ophthalmol Vis Sci 43, 2806-2814.
18. Kommonen, B., Dawson, W. W., and Parmer, R. (1991) Pigment Epithelial Function in Canine Retina, Am J Vet Res 52, 1341-1344.
19. Li, Z., Kapan, M. L., and Hachey, D. L. (2000) Hepatic Microsomal and Peroxisomal Docosahexaenoate Biosynthesis During Piglet Development, Lipids 35, 1325-1333.
20. Litman, B. J., Niu, S. L., Polozova, A., and Mitchell, D. C. (2001) The Role of Docosahexaenoic Acid Containing Phospholipids in Modulating G Protein-Coupled Signaling Pathways, J Molec Neurosci 16, 237-242.
21. Martinez, M. (1992) Tissue Levels of Polyunsaturated Fatty Acids During Early Human Development, J Pediatr 120, S129-138.
22. Menard, C. R., Goodman, K. J., Corso, T. N., Brenna, J. T., and Cunnane, S. C. (1998) Recycling of Carbon Into Lipids Synthesized de Novo is a Quantitatively Important Pathway of Alpha-[U-13C]Linolenate Utilization in the Developing Rat Brain, J Neurochem 71, 2151-2158.
23. Neuringer, M., Conner, W. E., Van Paten, C., and Barstow, L. (1984) Dietary omega-3 fatty acid deficiency and visual loss in infant rhesus monkeys,. J. Clin. Invest. 73, 272-76.
24. Pawlosky R. J., Denkins, Y., Ward, G. et al. (1997) Retinal and brain accretion of long-chain polyunsaturated fatty acids in developing felines: The effects of corn-based maternal diets. Am. J. Nutr. 665, 465-472.
25. Rotstein, N. P., Aveldano, M. I., and Politi, L. E. (1999) Essentiality of docosahexaenoic acid in retina photoreceptor cell development. Lipids 34, S115.
26. Sinclair, A. J. (1975) Long Chain Polyunsaturated Fatty Acids in the Mammalian Brain, Proc. Nutr. Soc. 34, 287-291.
27. Uauy, R., Hoffman, D. R., Mena, P., Llanos, A., and Birch, E. E. (2003) Term infant studies of DHA and ARA supplementation on neurodevelopment: results of randomized controlled trials, J Pediatr. 143, 17-25.
28. Willats, P. (2000) Long chain polyunsaturated fatty acids improve cognitive development. J. Fam. Health Care. 12, 5.

The present invention is not limited to the embodiments described and exemplified above, but is capable of variation and modification within the scope of the appended claims.

What is claimed:

1. A dietary composition comprising one or more long chain polyunsaturated fatty acids (LCPUFA), in an amount effective for improving visual acuity in an animal.

2. The composition of claim 1, wherein the animal is a companion animal.

3. The composition of claim 2, wherein the companion animal is a dog or cat.

4. The composition of claim 1 wherein the LCPUFA include at least one of arachidonic acid, linoleic acid, eicosapentaenoic acid, docosapentaenoic acid, or docosahexaenoic acid.

5. The composition of claim 1, wherein the LCPUFA are present in an amount of at least about 0.1% to about 10% by weight of the composition.

6. The composition of claim 1, wherein the LCPUFA are present in an amount of at least 1.8% to about 5.0% by weight of the composition.

7. The composition of claim 2, which is a pet food or a dietary supplement formulated for consumption by the companion animal.

8. The composition of claim 7, further comprising vitamins or minerals in amounts effective to promote health of the companion animal.

9. The composition of claim 7, further comprising one or more substances that sustain or promote ocular health or visual acuity in the companion animal.

10. The composition of claim 9, wherein the substances include one or more carotenoids, flavonoids or antioxidants.

11. The composition of claim 7, further comprising at least one type of probiotic organism.

12. A method for enhancing visual acuity in an animal comprising administering to the animal one or more LCPUFA in an amount effective to enhance visual acuity in the animal.

13. The method of claim 12, wherein the animal is a companion animal.

14. The method of claim 13, wherein the companion animal is a dog or cat.

15. The method of claim 12, wherein the LCPUFA include one or more of arachidonic acid, eicosapentaenoic acid, docosapentaenoic acid, or docosahexaenoic acid.

16. The method of claim 12, wherein the LCPUFA are administered to the animal during gestation.

17. The method of claim 12, wherein the LCPUFA are administered to the animal during the period spanning parturition through about twelve weeks after parturition.

18. The method of claim 12, wherein the LCPUFA are administered to the animal during gestation and during the period spanning parturition through about twelve weeks after parturition.

19. The method of claim 13, wherein the LCPUFA are administered in a pet food composition or a dietary supplement.

20. The method of claim 19 wherein the pet food composition or dietary supplement further comprises vitamins or minerals in amounts effective to promote health of the companion animal.

21. The method of claim 19 wherein the pet food composition or dietary supplement further comprises one or more substances that sustain or promote ocular health or visual acuity in the companion animal.

22. The method of claim 21 wherein the substances include one or more carotenoids, flavonoids or antioxidants.

23. The method of claim 19 wherein the pet food composition or dietary supplement further comprises at least one type of probiotic organism.

24. The method of claim 12, wherein the LCPUFA are administered in milk from a lactating animal to which has been administered one or more LCPUFA.

25. The method of claim 13, wherein the LCPUFA are administered in a pet food composition or dietary supplement and in milk from a lactating animal to which has been administered one or more LCPUFA.

26. The method of claim 12, wherein the LCPUFA are administered to the animal on a daily basis.
